(19) United States
(12) Patent Application Publication
(10) Pub. No.: US 2022/0096071 A1
(43) Pub. Date: Mar. 31, 2022

(54) SURGICAL RETRACTOR INCLUDING NON-CONDUCTIVE MEMBRANE

(71) Applicant: Covidien LP, Mansfield, MA (US)

(72) Inventors: Jacob C. Baril, Norwalk, CT (US); Justin J. Thomas, New Haven, CT (US); Garrett P. Ebersole, Hamden, CT (US); Saumya Banerjee, Collinsville, CT (US); Roy J. Pilletere, Middletown, CT (US); Matthew A. Dinino, Newington, CT (US)

(21) Appl. No.: 17/468,796

(22) Filed: Sep. 8, 2021

Related U.S. Application Data
(60) Provisional application No. 63/084,182, filed on Sep. 28, 2020.

(51) Int. Cl. A61B 17/02

(52) U.S. Cl. CPC A61B 17/0206; A61B 2017/00371; A61B 17/0218

(57) ABSTRACT

A surgical retractor has a shaft having a proximal end portion and a distal end portion. A handle assembly is coupled to the proximal end portion of the shaft and an end effector is coupled to the distal end portion of the shaft. The end effector includes a retractor, a first arm is configured to articulate the end effector and a second arm configured to transition the retractor between an expanded state and a collapsed state.

### CROSS-REFERENCE TO RELATED APPLICATIONS

The present application claims priority to U.S. Provisional Application No. 63/084,182, filed Sep. 28, 2020. The entire contents of which are incorporated herein by reference.

### FIELD

The present disclosure is generally related to a surgical retractor and more particularly to a surgical retractor having a non-conductive membrane for use in a minimally invasive surgical procedure.

### BACKGROUND

Minimally invasive surgical procedures including both endoscopic and laparoscopic procedures permit surgery to be performed on organs, tissues, and vessels far removed from an opening within the tissue. Surgical retractors are commonly used during minimally invasive surgical procedures to move or manipulate tissue and/or organs to provide space within a body cavity to perform the surgical procedure.

In some instances, during minimally invasive surgical procedures, electrocautery instruments may be used to treat tissue, however, these instruments may cause collateral damage to adjacent tissue (e.g., heating of tissue that is deeper than tissue being treated), unwanted stimulation of the nervous and/or muscle system, and/or possible malfunction of therapeutic devices in operative contact with a patient (e.g., a pacemaker).

### SUMMARY

In one aspect, the present disclosure provides a surgical retractor including a shaft, a handle assembly, and an end effector. The shaft has a proximal end portion and a distal end portion. The handle assembly is coupled to the proximal end portion of the shaft. The end effector is coupled to the distal end portion of the shaft. The end effector includes a retractor, a membrane disposed about the retractor, a first arm, and a second arm. The first arm has a distal end portion and a proximal end portion coupled to the distal end portion of the shaft, wherein the first arm is configured to articulate the end effector. The second arm has a distal end portion and a proximal end portion wherein the second arm is coupled to the first arm via a link and the second arm is configured to transition the retractor coupled to the distal end of the second arm between an expanded state and a collapsed state.

In aspects, the retractor may include a plurality of fingers extending distally from the second arm and configured to pivot outwardly about a pivot point of the second arm.

In aspects, the plurality of fingers may be constructed from rigid material.

In aspects, the membrane may be disposed about the plurality of fingers of the retractor and configured to expand with the transition of the retractor between the expanded state and the collapsed state.

In aspects, the membrane may be constructed from a flexible non-conductive material.

In aspects, the end effector in the expanded state may be articulated about a joint disposed between the first arm and the second arm.

In aspects, the handle assembly may include a first button and a second button, wherein the first button is configured to articulate the end effector and the second button is configured to transition the retractor between the expanded state and the collapsed state.

In aspects, the surgical retractor may further include a first rod and a second rod extending distally from the handle assembly, wherein the first rod is coupled to the first button and the link and the second rod extending through the first arm is coupled to the second arm and the second button.

In aspects, actuation of the first button may translate the first rod proximally causing the first arm to articulate.

In aspects, actuation of the second button may translate the second rod causing the retractor to transition between the expanded state and the collapsed state.

In aspects, the second rod may be flexible to permit articulation of the first arm relative to the second arm.

In another aspect, a surgical retractor is disclosed having a handle with first and second sliders. A shaft extends from the handle. A first arm is disposed at a distal portion of the shaft and is operatively coupled to the first slider via a first rod and configured to articulate relative to the shaft in response to actuation of the first slider. A second arm is disposed about the first arm and is operatively coupled to the second slider via a second rod. A retractor is disposed at a distal portion of the second arm and includes blades. The blades transition between a collapsed state and an expanded state in response to actuation of the second slider. A membrane is disposed about the blades.

The details of one or more aspects of the surgical retractor in accordance with the present disclosure are set forth in the accompanying drawings and the description below. Other features, objects, and advantages of the surgical access assemblies described in this disclosure will be apparent from the description and drawings, and from the claims.

### BRIEF DESCRIPTION OF DRAWINGS

FIG. 1 is a perspective view an exemplary surgical retractor according to the present disclosure;

FIG. 2 is an exploded perspective view, with parts separated, of the surgical retractor of FIG. 1;

FIG. 3 is an enlarged view of the area of detail indicated in FIG. 2;

FIG. 4 is an end cross-sectional view taken along section line 4-4 of FIG. 1;

FIG. 5 is a perspective view of the surgical retractor of FIG. 1 with an end effector of the surgical retractor proximate tissue and in a collapsed state;

FIG. 6 is an enlarged view of the area of detail indicated in FIG. 5 showing a membrane disposed about the end effector;

FIG. 7 is a top view of the end effector of the surgical retractor of FIG. 6 without the membrane;

FIG. 8 is a side cross-sectional view taken along section line 8-8 of FIG. 5 with the end effector of the surgical retractor in the collapsed state and the membrane removed;

FIG. 9 is a perspective view of the surgical retractor of FIG. 1 with the surgical retractor proximate tissue and the end effector in an expanded state;

FIG. 10 is an enlarged view of the area of detail indicated in FIG. 9;

FIG. 11 is a top view of the end effector of the surgical retractor of FIG. 10 with the membrane removed;

FIG. 12 is a perspective view of a surgical instrument and the surgical retractor of FIG. 1 with the end effector positioned directly behind tissue; and

FIG. 13 is a side cross-sectional view taken along section line 13-13 of FIG. 12.

### DETAILED DESCRIPTION

Aspects of the presently disclosed surgical retractor are described in detail with reference to the drawings, in which like reference numerals designate identical or corresponding elements in each of the several views. As used herein, the term “distal” refers to that portion of the surgical retractor or component thereof, farther from the user, while the term “proximal” refers to that portion of the surgical retractor, or component thereof, closer to the user.

As used herein, the term “clinician” refers to a doctor, nurse, surgeon, or other care provider and may include support personnel. In the following description, well-known functions, or construction are not described in detail to avoid obscuring the disclosure in unnecessary detail.

In general, the present disclosure provides a surgical retractor for moving and manipulating tissue within a patient having a non-conductive membrane disposed about an end effector of the surgical retractor.

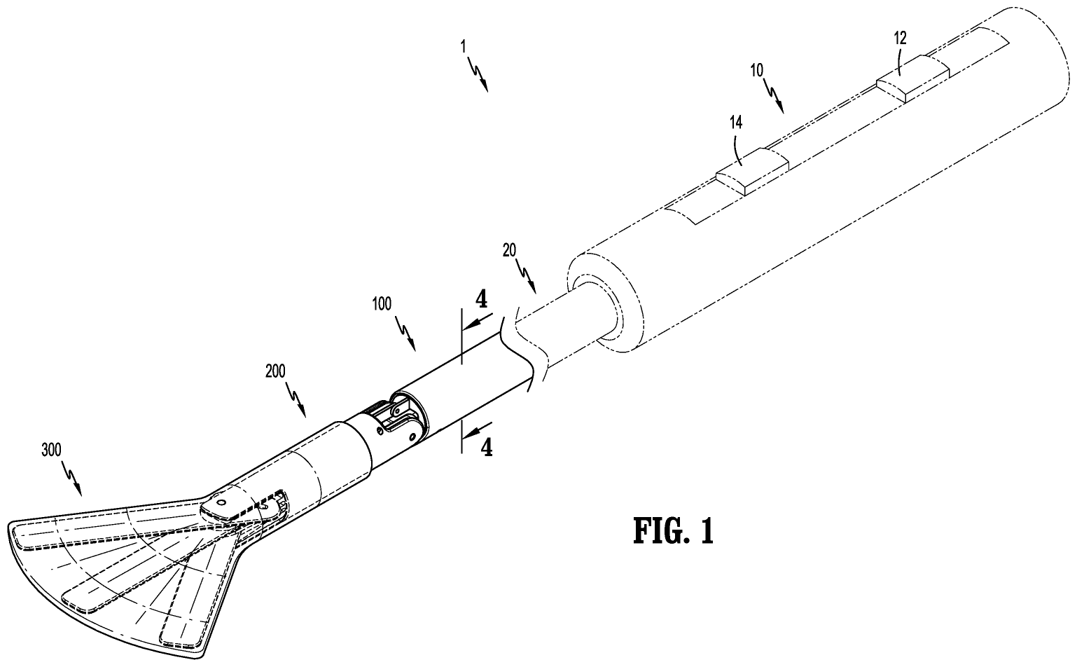

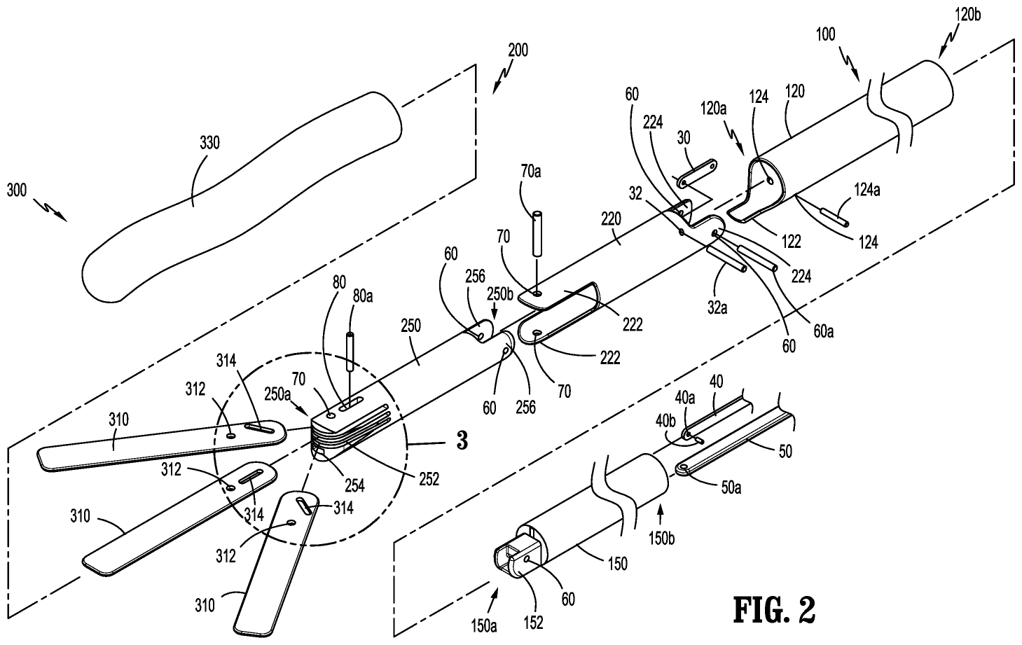

Referring initially to FIGS. 1 and 2, a surgical retractor 1 generally includes a handle assembly 10, a shaft 20 extending distally from the handle assembly 10, and an end effector 300 extending distally from the shaft 20. The surgical retractor 1 further includes an articulation arm 100 and a retraction arm 200 operably coupled to the articulation arm 100 that is disposed between a distal end portion of the shaft 20 and the end effector 300. The retraction arm 200 surrounds the articulation arm 100.

The articulation arm 100 generally includes an articulation outer sleeve 120 and an articulation member 150. The articulation member 150 has a distal end portion 150a and a proximal end portion 150b. With additional reference to FIG. 4, the articulation member 150 includes a first aperture 154 and a second aperture 156 extending distally from the proximal end portion 150b of the articulation member 150 to the distal end portion 150a of the articulation member 150. The first aperture 154 of the articulation member 150 is sized to slidably receive an articulation rod 40 and the second aperture 156 is sized to slidably receive a drive rod 50. The drive rod 50 is constructed from a flexible material (e.g., laminate). The articulation rod 40 is constructed from a rigid material. The articulation member 150 includes an elbow 152 fixedly coupled to the distal end portion 150a of the articulation member 150. The elbow 152 has an opening (not shown) extending distally through the elbow 152 that is configured to align with the second aperture 156 of the articulation member 150.

The articulation outer sleeve 120 includes a lip 122 extending from a distal end portion 120a of the articulation outer sleeve 120 and configured to align with an elbow 152 of the articulation member 150. The elbow 152 of the articulation member 150 and the lip 122 of the articulation outer sleeve 120 are configured to prevent hyperextension of the retraction arm 200. The articulation outer sleeve 120 is coaxially mounted over the articulation member 150 via an articulation coupling member 124a (e.g., a pin) inserted through an articulation mounting aperture 124 extending through the articulation outer sleeve 120 and the articulation member 150 (not shown).

The retraction arm 200 generally includes a retraction outer sleeve 220 and a retraction member 250. The retraction member 250 has a distal end portion 250a defining a plurality of slits 252 and a proximal end portion 250b. The plurality of slits 252 is configured to receive a plurality of blades or fingers 310 of the end effector 300. The proximal end portion 250b of the retraction member 250 include guides 256 extending proximally from the retraction member 250 and configured to guide movement of the retraction arm 200 along an outer surface of the elbow 152 of the articulation arm 100.

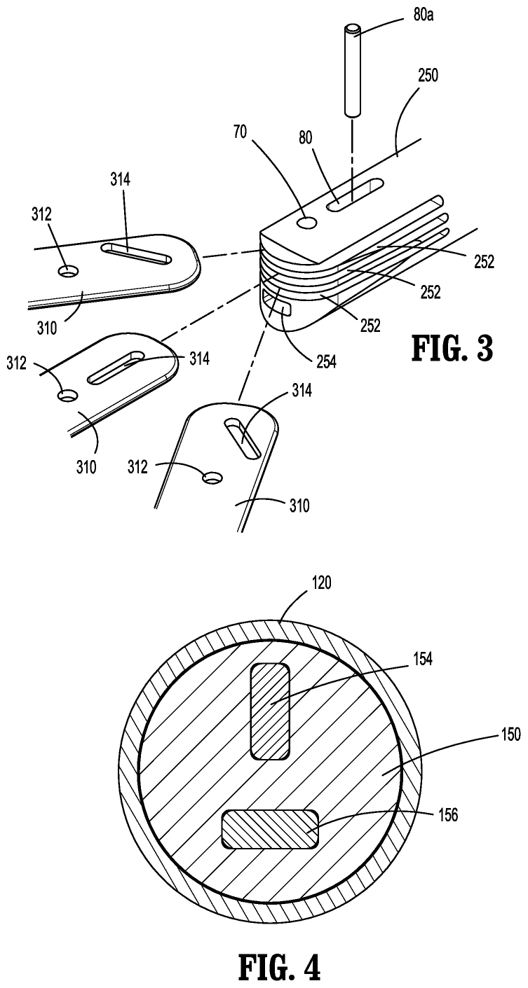

With additional reference to FIG. 3, the retraction member 250 includes a drive rod channel 254 extending distally from a distal end portion 250a to a proximal end portion 250b sized to slidably receive the drive rod 50. The retraction member 250 further includes a pivot aperture 70 and a retraction slot 80. The pivot aperture 70 and the retraction slot 80 extend from a top surface of the retraction member 250 to a bottom surface of the retraction member 250 and through the plurality of slits 252.

Referring now to FIG. 2, the retraction outer sleeve 220 of the retraction arm 200 defines a tube including prongs or fingers 222 extending distally from a top and bottom surface of the retraction outer sleeve 220 and prongs or fingers 224 extending proximally from side surfaces of the retraction outer sleeve 220. The fingers 222 are configured to align with the distal end portion 250a of the retraction member 250 and the fingers 224 are configured to align with guides 256 of the retraction member 250. The pivot aperture 70 further extends through the fingers 222 of the retraction outer sleeve 220. The retraction outer sleeve 220 is coaxially mounted over the retraction member 250 via an pivot coupling member 70a (e.g., a pin) inserted through the pivot aperture 70 extending through the fingers 222 of the retraction outer sleeve 220 and the plurality of slits 252 of the retraction member 250.

With additional reference to FIG. 8, the articulation arm 100 is operably coupled to the retraction arm 200 via an arm coupling member 60a inserted into an arm coupling aperture 60 extending through the fingers 224 of the retraction outer sleeve 220, guides 256 of retraction member 250, and elbow 152 of the articulation member 150. The surgical retractor 1 further includes a link 30 disposed between articulation arm 100 and retraction arm 200. The link 30 includes a distal end aperture 30a and a proximal end aperture 30b. The proximal aperture 30b of the link 30 is configured to couple to an articulation rod aperture 40a and the distal end aperture 30a of the link 30 is configured to couple to the retraction arm 200 via link coupling member 32a (e.g., a pin) inserted into a link coupling aperture 32 disposed on the outer surface of the retraction arm 200.

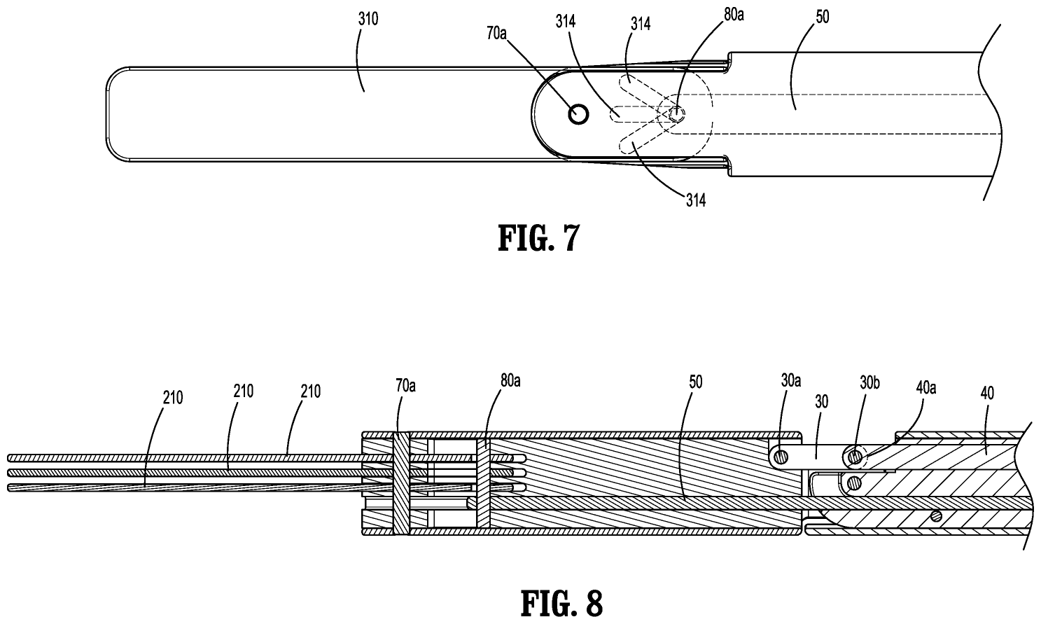

Referring also to FIGS. 2 and 3, each finger 310 of the plurality of fingers 310 of the end effector 300 includes a slot 314 and an aperture 312. Each slot 314 is configured be angled in a predetermined direction such that when the plurality of fingers 310 is in a collapsed state (FIG. 7), a proximal end portion of each slot 314 is aligned with the retraction slot 80 of the retraction member 250. Each slot 314 is angled in the predetermined direction such that translation of the drive rod 50 drives movement of each of the finger 310 of the plurality of fingers 310 in the predetermined direction based on the angle of the slot 314. Each aperture 312 is configured to align with the pivot aperture 70 of the retraction member 250 to provide a pivot point for each finger 310 of the plurality of fingers 310 about the pivot aperture 70. The plurality of fingers 310 of the end effector 300 is operably coupled to the retraction arm 200 via a drive pin 80a and a pivot pin 70a. The drive pin 80a is inserted through the retraction slot 80 of the retraction member 250, the slots 314 of the fingers 310, and the drive rod aperture 50a of the drive rod 50 which extends from the handle assembly 10 to the retraction member 250 through the drive rod channel 254. The pivot pin 70a is inserted through the pivot aperture 70 extending through the retraction outer sleeve 220, retraction member 250, the apertures 312 of the plurality of fingers 310, and the plurality of slits 252. The fingers 310 may be constructed of a rigid material.

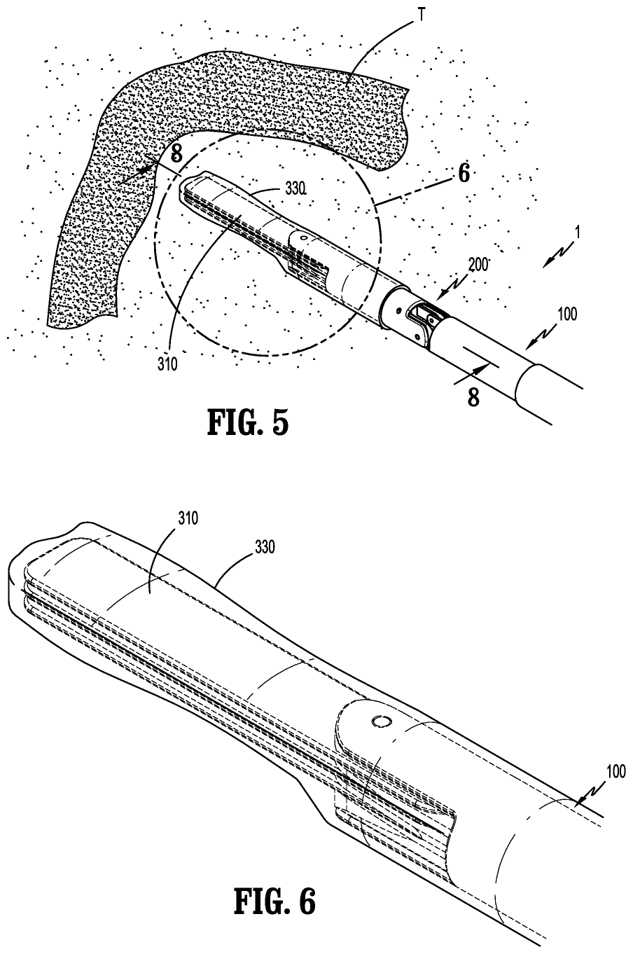

With reference now to FIGS. 5 and 6, the end effector 300 further includes a membrane 330 configured to receive the plurality of fingers 310 of the end effector 300 (FIG. 6). The membrane 330 is configured to expand and accommodate the plurality of fingers 310 of the end effector 300 upon expansion of the plurality of fingers 310 of the end effector 300 in all directions. The membrane 330 is constructed of an electrically non-conductive plastic or other material with non-conductive properties. The plurality of fingers 310 receives the membrane 330 and the membrane 330 is securely fitted around the retraction arm 200 via expansion of the membrane 330 over a distal end portion of the retraction arm 200. Additionally, and/or alternatively, the membrane 330 may be fitted around the retraction arm 220 via expansion of the membrane 330 over each finger 310 of the plurality of fingers 310 of the end effector and subsequently the distal end portion of the retraction arm 200.

Referring back to FIG. 1, the handle assembly 10 of the surgical retractor 1 includes a retraction slider 12 and an articulation slider 14. The retraction slider 12 is operatively coupled to the drive rod 50 and is configured to transition the plurality of fingers 310 of the end effector 300 between an expanded state and a collapsed state (FIG. 7). The articulation slider 14 is operatively coupled to the articulation rod 40 and is configured to actuate the retraction arm 200 and the end effector 300 relative to articulation arm 100 about the pivot coupling member 70a (e.g., a pin). Either slider 12, 14 may be any suitable mechanical control e.g., levers, wheels, triggers, buttons, etc. Additionally and/or alternatively, the retraction slider 12 and the articulation slider 14 may be disposed in any suitable location along the handle assembly for ease of use by the clinician, such as, for example switching locations of the retraction slider 12 with that of the articulation slider 14.

In operation, the surgical retractor 1 may be used in a minimally invasive surgery to retract tissue and/or organs for improved visibility in the surgical site and accessibility to the surgical site. The surgical retractor 1 is advanced into the patient towards the surgical site while the end effector 300 is in a collapsed state, until the end effector 300 engages a tissue “T” (FIG. 5).

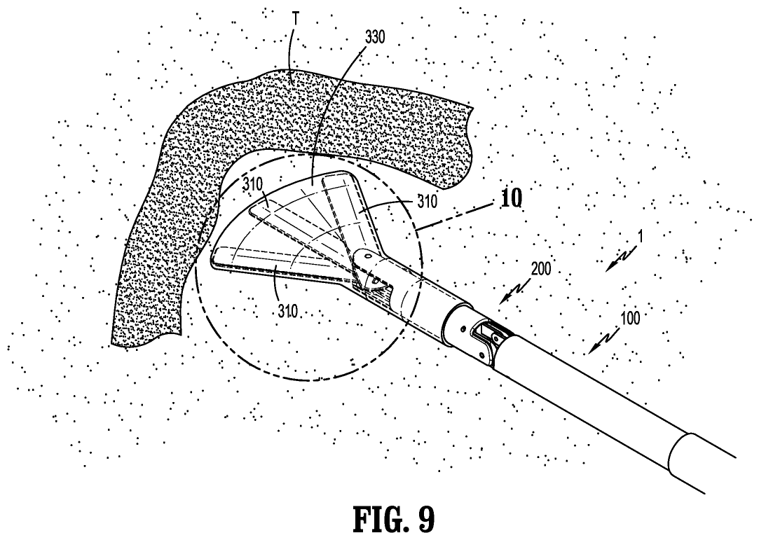

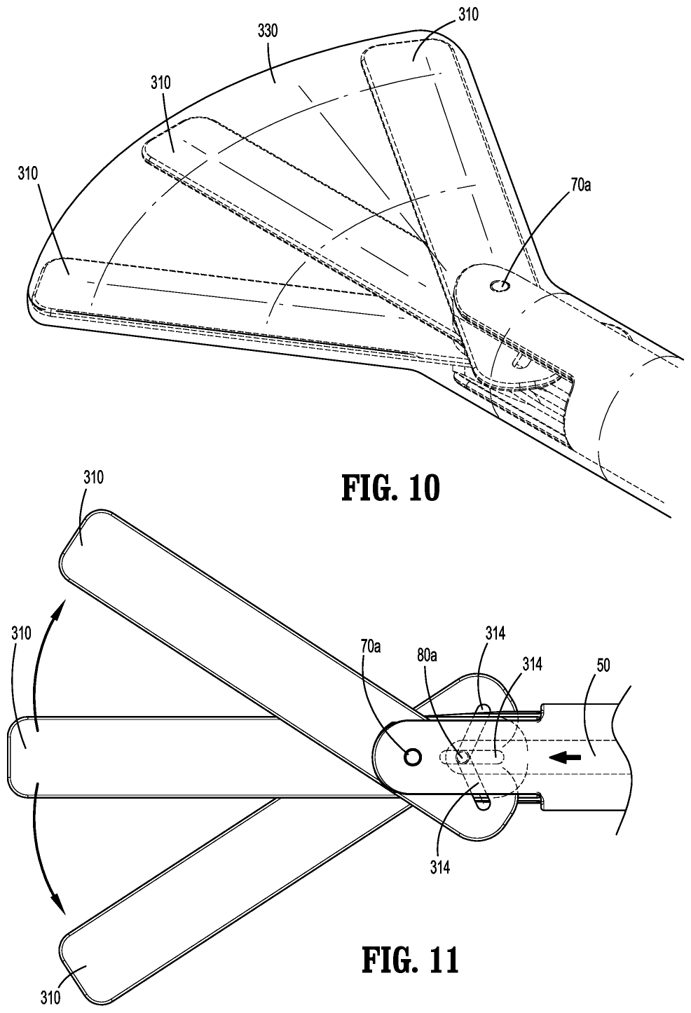

Referring to FIGS. 7-9, upon positioning of the surgical retractor 1 to engage the tissue “T”, the retraction slider 14 is actuated moving the drive bar 50 distally from a first position (FIGS. 7 and 8) to a second position (FIG. 11). Once the drive bar 50 is slid from the first position to the second position, the drive pin 80a coupled to the drive bar 50 advances distally from the first position to the second position (FIG. 11) thereby causing at least one of the plurality of fingers 310 to pivot outwardly about the pivot coupling member 70a and transitioning the end effector 300 from the collapsed state to an expanded state (FIG. 11). Upon transitioning the end effector 300 from the collapsed state to the expanded state, the membrane 330 is expanded, and the tissue “T” is manipulated.

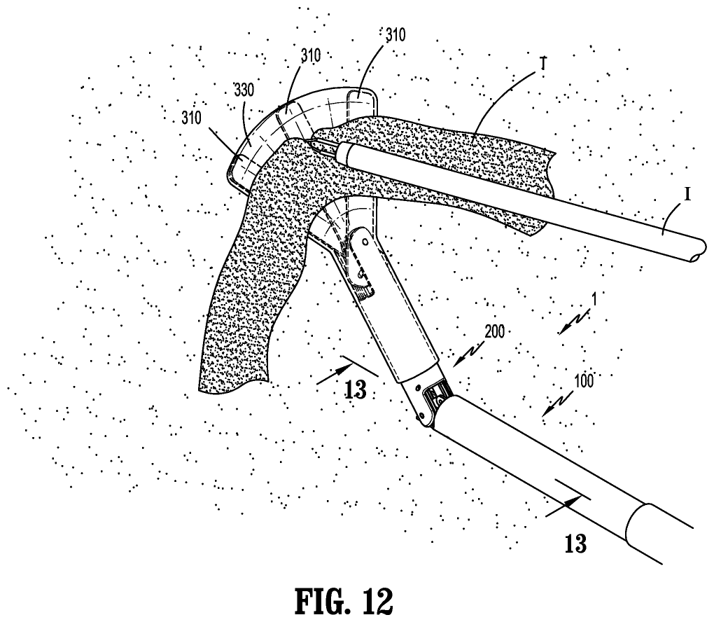

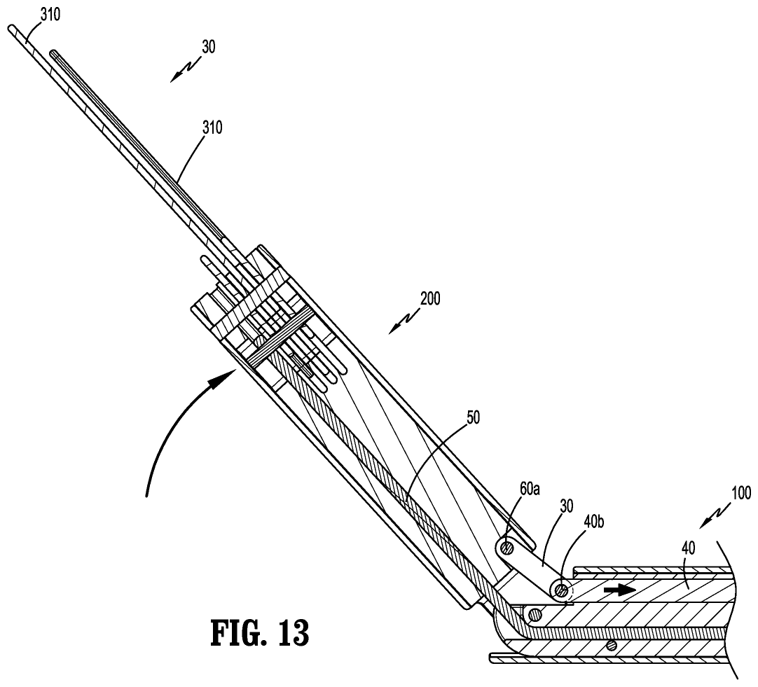

With further reference to FIGS. 12 and 13, the surgical retractor 1 may be further positioned directly behind the tissue “T”, while the end effector 300 of the surgical retractor 1 is in the expanded state to provide a backstop for tissue dissection via a surgical instrument utilizing electrical energy. Once the surgical retractor is advanced past the tissue “T”, the articulation slider 12 is actuated thereby causing the articulation rod 40 to move proximally towards the handle assembly 10 (FIG. 13). As the articulation rod 40 moves proximally, the link 30 coupled to the articulation rod 40 and the retraction arm 200 is pulled proximally thereby causing the retraction arm 200 and the end effector 300 to pivot inwardly about the arm coupling member 60a away from the lip 122 of the articulation arm 100. Upon positioning of the end effector 300 of the surgical retractor 1 in the expanded state directly behind the tissue “T”, the tissue “T” is isolated from the surrounding tissues and the surgical instrument utilizing electrical energy such as, for example, an electrocautery instrument “I” is advanced into the patient towards the surgical site to manipulate the tissue “T” (FIG. 12). The surgical retractor 1 positioned behind the tissue “T” during use of the electrocautery instrument “I” minimizes the likelihood of collateral tissue damage resulting from thermal spreading.

It should be understood that various features of the surgical retractor specifically disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques).

What is claimed is:

1. A surgical retractor comprising:
a shaft having a proximal end portion and a distal end portion;
a handle assembly coupled to the proximal end portion of the shaft; and
an end effector coupled to the distal end portion of the shaft, the end effector including:
a retractor;
a membrane disposed about the retractor;
a first arm having a distal end portion and a proximal end portion coupled to the distal end portion of the shaft, wherein the first arm is configured to articulate the end effector; and
a second arm having a distal end portion and a proximal end portion wherein the second arm is coupled to the first arm via a link and the second arm is configured to transition the retractor coupled to the distal end of the second arm between an expanded state and a collapsed state.

2. The surgical retractor of claim 1, wherein the retractor further includes a plurality of fingers extending distally from the second arm and configured to pivot outwardly about a pivot point of the second arm.

3. The surgical retractor of claim 2, wherein the plurality of fingers is constructed from rigid material.

4. The surgical retractor of claim 2, wherein the membrane is disposed about the plurality of fingers of the retractor and configured to expand with the transition of the retractor between the expanded state and the collapsed state.

5. The surgical retractor of claim 4, wherein the membrane is constructed from a flexible non-conductive material.

6. The surgical retractor of claim 1, wherein the end effector in the expanded state is articulated about a joint disposed between the first arm and the second arm.

7. The surgical retractor of claim 1, wherein the handle assembly includes a first button and a second button, wherein the first button is configured to articulate the end effector and the second button is configured to transition the retractor between the expanded state and the collapsed state.

8. The surgical retractor of claim 7, further including a first rod and a second rod extending distally from the handle assembly, wherein the first rod is coupled to the first button and the link disposed between the first and second arm and the second rod extending through the first arm is coupled to the second arm and the second button.

9. The surgical retractor of claim 8, wherein actuation of the first button translates the first rod proximally causing the first arm coupled to the link to articulate.

10. The surgical retractor of claim 8, wherein actuation of the second button translates the second rod causing the retractor to transition between the expanded state and the collapsed state.

11. The surgical retractor of claim 8, wherein the second rod is configured to be flexible to permit articulation of the first arm relative to the second arm.

12. A surgical retractor comprising:
a handle having first and second sliders;
a shaft extending from the handle;
a first arm disposed at a distal portion of the shaft, the first arm operatively coupled to the first slider via a first rod and configured to articulate relative to the shaft in response to actuation of the first slider;
a second arm disposed about the first arm, the second arm operatively coupled to the second slider via a second rod;
a retractor disposed at a distal portion of the second arm, the retractor including blades, the blades transitioning between a collapsed state and an expanded state in response to actuation of the second slider; and
a non-conductive membrane disposed about the blades.
